(19) United States
(12) Patent Application Publication
(10) Pub. No.: US 2012/0251482 A1
(43) Pub. Date: Oct. 4, 2012

(54) USE FOR IMPROVING 5-HT FUNCTION AND ENOS EXPRESSION OF KMUPS AMINE SALTS

(75) Inventor: Ing-Jun Chen, Kaohsiung (TW)

(73) Assignee: KAOHSIUNG MEDICAL UNIVERSITY, Kaohsiung (TW)

(21) Appl. No.: 13/235,971

(22) Filed: Sep. 19, 2011

(30) Foreign Application Priority Data
Mar. 30, 2011 (TW) 100111094

(51) Int. Cl. A61K 31/787; C07K 2/00; C08B 37/04; C08B 37/02; C08B 37/10; C08B 37/08; C07D 473/08; A61K 31/522; A61P 9/12; A61P 9/00; A61K 38/02; A61K 31/717; A61K 31/734; A61K 31/727; A61K 31/721; A61K 31/728; C08F 26/06; C08B 15/06

(52) U.S. Cl. 424/78.29; 536/31; 530/350; 536/3; 536/51; 536/21; 536/55.1; 544/270; 514/252.16; 514/1.1; 514/57; 514/54; 514/59; 514/56; 526/259

(57) ABSTRACT

The synthesized piperazium salt of KMUPs disclosed in the present invention is characterized by presented pharmaceutics having functions to improve 5-HT function and eNOS expression of KMUPS in lung diseases, such as proliferation, obliteration, pulmonary artery hypertension. The pharmaceutical composition for inhibiting monocrotaline (MCT)-induced proliferation of pulmonary artery includes an effective amount of a complex salt of formula (I):

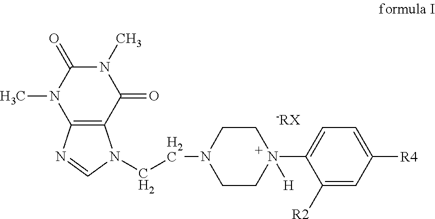

wherein R2 and R4 are each selected independently from the group consisting of a C1˜C5 alkoxy group, a hydrogen, a nitro group, and a halogen atom; RX contains a carboxylic group donated from one selected from a group consisting of a Statin, a Co-polymer, a poly-γ-polyglutamic acid (γ-PGA) derivative and sodium CMC; and
⁻RX substituent is an anion of the carboxylic group carrying a negative charge; and a pharmaceutically accepted carrier.

### CROSS-REFERENCE TO RELATED APPLICATIONS

This application claims the benefit of Taiwan Application No. 100111094 filed Mar. 30, 2011 the contents of which is incorporated herein by reference in its entirety.

### FIELD OF THE INVENTION

The present invention relates to KMUPs amine salts compound capable of enhancing the pulmonary vascular endothelial cells eNOS expression. In particular, the KMUPs amine salts inhibiting monocrotaline (MCT)-induced proliferation of pulmonary artery smooth muscle cells through 5-HT, 5-HT receptors and serotonin transporter (5-HTT).

### BACKGROUND OF THE INVENTION

Serial developed KMUP derivatives comprised of theophylline base linked with a piperazinyl group and showed the pleitropic activity. The present invention provides KMUP amine salts synthesized by the KMUP compounds and a carboxylic acid moiety of one selected from a group consisting of a statin, a non-steroid anti-inflammatory (NSAIDs) and an anti-asthmatic drug. The pharmaceutical composition for the treatment of an interstitial lung disease have been applied as Ser. No. 11/857,483 filed on Sep. 19, 2007.

A KMUP derivative, 7-[2-[4-(2-chlorophenyl)piperazinyl]ethyl]-1,3-dimethyl-xanthine, KMUP-1, with a nitrogen group at the 7^{th} position of theophylline base could induce release of endogenous nitric oxide and had a similar pharmacological mechanism of action to a nitric oxide (NO) donor. Therefore, Taiwan Applications No. 094129421 and No. 096121950 were applied with the relevant mechanism of action.

Proliferation and obliteration are main symptoms of a pulmonary artery hypertension. There are many pathogenesis of the pulmonary artery hypertension, including the proliferation caused by serotonin (5-hydroxytryptamine, 5-HT)-induced expression of 5-HT transporter (5-HTT) and activation of Rhodostomin A (RhoA).

5-HT internalized in smooth muscle through the 5-HTT is covalently linked to RhoA by intracellular type 2 transglutaminase, leading to constitutive RhoA activation in pulmonary artery of pulmonary artery hypertension. RhoA and Rho kniase (ROCK) controls a wide variety of signal transduction pathways. In vascular system, RhoA mediates vascular constriction via inhibiting phosphorylation of myosin light chains and myosin phosphatase.

5-HT promotes mitosis of pulmonary arterial smooth muscle cell (PASMC) that is important to pulmonary artery remodeling. 5-HT activates 5-HTT via binding to at least one of 5-HT_{2A}, 5-HT_{2B}, 5-HT_{2C} receptors to trigger the cell mitosis. 5-HT_{2B} receptors of endothelium can be stimulated to express eNOS and release NO, leading increasing cGMP of vascular smooth muscle cells to inhibit vascular constriction induced by the agonist 5-HT. Therefore, 5-HT_{2B} receptors mediate vascular relaxation of pulmonary artery.

An extracellular signal-regulated kinase (ERK) 1/2 and a Serine/Threonine kinase (AKT) pathways are required for 5-HT to induce mitosis. AKT signaling plays important roles in vascular smooth muscles (VSMCs) mediating cell survival, proliferation and the migration of VSMCs induced by 5-HT. Intracellular [Ca²⁺]i in the smooth muscle cells of the pulmonary artery can be affected by 5-HT. Intracellular [Ca²⁺]i activates the myosin light chain kinase to make a constriction of vascular smooth muscles, and is the main factor for regulating the proliferation of the pulmonary artery. Furthermore, 5-HT reacts with c-fos gene or c-jun gene to activate Ca²⁺/cAMP response element binding protein (CREB) and mitogen-activated protein kinase (MAPK) and increase the intracellular [Ca²⁺]i to cause cell proliferation.

### SUMMARY OF THE INVENTION

In accordance with an aspect of the present invention, the complex compound comprising a structure being one of formula I:

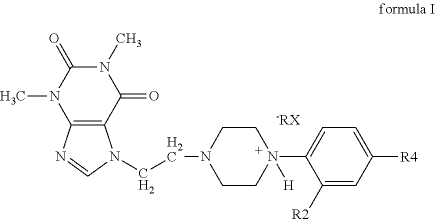

Wherein R2 and R4 are each selected independently from the group consisting of a C1˜C5 alkoxy group, a hydrogen, a nitro group, and a halogen atom;
RX contains a carboxylic group which donated from one of a Statin, a Co-polymer, a poly-γ-polyglutamic acid (γ-PGA) derivative and sodium carboxyl methylcellulose (sodium CMC); and
⁻RX substituent is an anion of the carboxylic group carrying a negative charge; and
halogen atom is one selected from a group consisting of a fluorine, a chlorine, a bromine and an iodine.

In accordance with another aspect of the present invention, an pharmaceutical composition is provided. The pharmaceutical composition includes:

an effective amount of a compound of formula I:

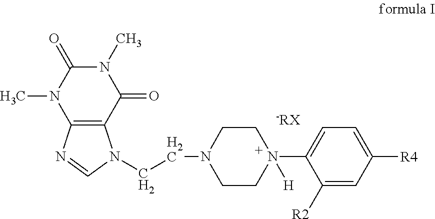

Wherein R2 and R4 are each selected independently from the group consisting of a C1˜C5 alkoxy group, a hydrogen, a nitro group, and a halogen atom;
RX contains a carboxylic group which donated from one of a Statin, a Co-polymer, a poly-γ-polyglutamic acid derivative and sodium CMC; and
⁻RX substituent is an anion of the carboxylic group carrying a negative charge, halogen atom is one selected from a group consisting of a fluorine, a chlorine, a bromine and an iodine; and
a pharmaceutically acceptable carrier.

In accordance with a further aspect of the present invention, complex compounds, characteristically with inhibition activity on proliferation of monocrotaline-induced pulmonary artery, is provided. The complex compound comprises a common structure being the following formula I:

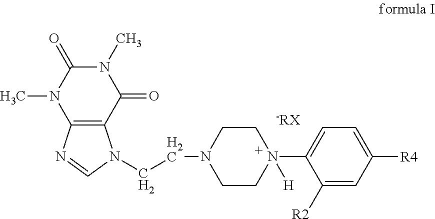

Wherein R2 and R4 are each selected independently from the group consisting of a C1˜C5 alkoxy group, a hydrogen, a nitro group, and a halogen atom;
RX contains a carboxylic group which donated from one of a Statin, a Co-polymer, a poly-γ-polyglutamic acid derivative and sodium CMC; and
⁻RX substituent is an anion of the carboxylic group carrying a negative charge; and
halogen atom is one selected from a group consisting of a fluorine, a chlorine, a bromine and an iodine.

In accordance with a further aspect of the present invention, pharmaceutical composition of complex compounds, characteristically with inhibition activity on monocrotaline-induced pulmonary artery proliferation is provided. The pharmaceutical composition includes an effective amount of a compound of formula I:

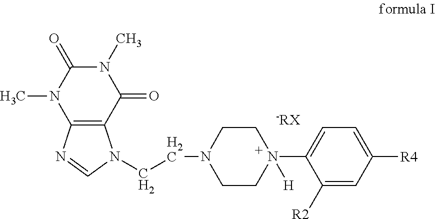

Wherein R2 and R4 are each selected independently from the group consisting of a C1˜C5 alkoxy group, a hydrogen, a nitro group, and a halogen atom;
RX contains a carboxylic group which donated from one of a Statin, a Co-polymer, a poly-γ-polyglutamic acid derivative and sodium CMC; and
RX⁻ substituent is an anion of the carboxylic group carrying a negative charge, halogen atom is one selected from a group consisting of a fluorine, a chlorine, a bromine and an iodine; and a pharmaceutically acceptable carrier.

The above objects and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 is a diagram illustrating that monocrotaline induces the amount of 5-HT in rats plasma
A: monocrotaline (MCT)
B: 5 mg/kg p.o.
C: 1 mg/kg i.p.
D: KMUP-1 and MCT
^{#}P<0.05 MCT group compared with the control group
*P<0.05 MCT group compared with the KMUP-1

FIG. 2 is a diagram illustrating the inhibition of pulmonary artery constriction
A: 5-HT and KMUP-1
B: 5-HT, KMUP-1 and _{L}-NAME
C: 5-HT and simvastatin
*P<0.05, KMUP-1 and _{L}-NAME compared with KMUP-1

FIG. 3 is a diagram illustrating [Ca²⁺]i of pulmonary artery smooth muscle cells
A: 5-HT (10 μM)
B: 5-HT and KMUP-1 (0.1 μM)
C: 5-HT and KMUP-1 (1 μM)
D: 5-HT and KMUP-1 (10 μM)
E: 5-HT and KMUP-1 (100 μM)

FIG. 4 is a diagram illustrating the Δ[Ca²⁺]i (the difference in [Ca²⁺]i) of pulmonary artery smooth muscle cells
A: 5-HT (10 μM) and KMUP-1
*P<0.05, **P<0.01 5-HT compared with KMUP-1

FIG. 5 is a diagram showing percentage expression of 5-HT after MCT between control and KMUP-1 treatment.
A: control group
B: MCT
C: 5 mg/kg p.o.
D: 1 mg/kg i.p.
F: KMUP-1 and MCT
^{#}P<0.05 compared with control;
*P<0.05, *P<0.01 compared with MCT

FIG. 6 is a picture showing time course of 5-HT (10 μM) induced 5-HTT expression

FIG. 7 is a diagram illustrating percentage expression of 5-HTT
A: KMUP-1
B: Y27632
^{##}P<0.01 compared with the control group
*P<0.05, **P<0.01 compared with 5-HT treated cells

FIG. 8 is a diagram illustrating percentage of 5-HTT expression
A: control group
B: 5-HT group
C: KMUP-1
D: simvastatin
^{##}P<0.01 compared with the control group
*P<0.05 compared with 5-HT treated cells

FIG. 9 is a diagram illustrating time courses of 5-HT-induced RhoA translocation
^{##}P<0.01, ^{#}P<0.05 compared with the untreated control group

FIG. 10 is a diagram illustrating time courses of ROCK expression in pulmonary arterial smooth muscle cells (PASMCs)
^{#}P<0.05 compared with the untreated control group

FIG. 11 is a diagram illustrating RhoA activation of membrane and cytosol
A: 5-HT group
B: KMUP-1
C: Y27632
^{#}P<0.01 compared with the control group
*P<0.05, **P<0.01 compared with 5-HT

FIG. 12 is a diagram illustrating the expression of ROCK
A: 5-HT
B: KMUP-1
C: Y27632
*P<0.05, **P<0.01 compared with 5-HT
^{#}P<0.05, compared with the control group

FIG. 13 is a diagram illustrating time courses of p-ERK1/2
^{#}P<0.05, ^{##}P<0.01 compared with the control group

FIG. 14 is a diagram illustrating time courses of p-AKT
^{#}P<0.01 compared with the control group

FIG. 15 is a diagram illustrating the ratio of p-ERK1/2
A: control group
C: KMUP-1 and 5-HT (10 μM)
^{#}P<0.01 compared with the control group
*P<0.05, **P<0.01 compared with 5-HT

FIG. 16 is a diagram illustrating the ratio of p-AKT
A: control group
C: KMUP-1 and 5-HT (10 μM)
^{##}P<0.01 compared with the control group
*P<0.05 compared with 5-HT

FIG. 17 is a diagram illustrating migration of PASMCs
A: control group
B: 5-HT
C: KMUP-1
D: simvastatin
^{##}P<0.01 compared with the control group
*P<0.05, **P<0.01 compared with 5-HT

FIG. 18 is a diagram illustrating proliferation rate of PASMCs
A: 5-HT
B: KMUP-1
C: simvastatin
^{##}P<0.01 compared with the control group
*P<0.05, **P<0.01 compared with 5-HT

FIG. 19 is a diagram showing the expression of eNOS and 5-HT_{2B} receptor
A: control group
B: KMUP-1
*P<0.05, **P<0.01 compared with the control group

FIG. 20 is a diagram illustrating the expression of eNOS
A: control group
B: 5-HT
C: 5-HT and KMUP-1
^{#}P<0.05 compared with the control group;
*P<0.05, **P<0.01 compared with the 5-HT treated HPAEC.

FIG. 21 is a diagram illustrating the production of NO
A: control group
B: 5-HT
C: 5-HT and KMUP-1 ^{##}P<0.01 compared with the control group;
**P<0.01 compared with the 5-HT treated human pulmonary artery endothelial cell (HPAEC)

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

A KMUPs amine salt having a formula I,

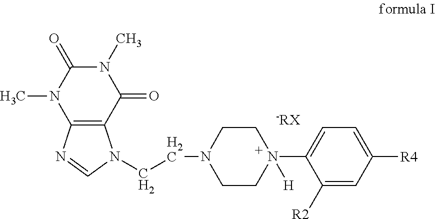

wherein R2 and R4 are each selected independently from the group consisting of a C1˜C5 alkoxy group, a hydrogen, a nitro group, and a halogen atom. The above-mentioned halogen refers to fluorine, chlorine, bromine and iodine. RX contains a carboxylic group which donated from a group consisting of a member of a Statin, a poly-γ-polyglutamic acid (γ-PGA) derivative and a sodium carboxyl methylcellulose (sodium CMC); ⁻Rx can be an anion of the above-mentioned groups carrying a negative charge.

Preferably, Statin is one selected from a group consisting of an Atorvastatin, a Cerivastatin, a Fluvastatin, a Lovastatin, a Mevastatin, a Pravastatin, Rosuvastatin and a Simvastatin. Co-polymers is one selected from a group consisting of a hyaluronic acid, a polyacrylic acid, a polymethacrylates (PMMA), an Eudragit, a dextran sulfate, a heparan sulfate, a polylactic acid or polylactide (PLA), a polylactic acid sodium (PLA sodium) and a polyglycolic acid sodium (PGA sodium). Poly-γ-polyglutamic acid (γ-PGA) derivative is one selected from a group consisting of an alginate sodium, a poly-γ-polyglutamic acid sodium (γ-PGA sodium), a poly-γ-polyglutamic acid (γ-PGA) and an alginate-poly-lysine-alginate (APA). Hyaluronic Acid is a polymer, which are composed of alternating units of N-acetyl glucosamine (NAG) and D-glucuronic acid. Eudragit is a trade name of series copolymers derived from esters of acrylic and methacrylate acid.

The term “KMUPs amine salt” as used herein refers to one selected from a group consisting of KMUP-1, KMUP-2, KMUP-3, KMUP-4 and its pharmaceutical acceptable salts. Preferably, the pharmaceutical composition further includes at least one of a pharmaceutically acceptable carrier and an excipient. Preferably, a theophylline-based moiety compound derivative, i.e. KMUPs, which is obtained by reacting theophylline compound with piperazine compound and is then recrystallized the intermediate therefrom, is provided in the present invention.

Preferably, in one embodiment, the compound of formula I is KMUP-1 amine salt, wherein R₂ is chlorine atom and R₄ is hydrogen atom, which has the generally chemical name 7-[2-[4-(2-chlorophenyl)piperazinyl]ethyl]-1,3-dimethylxanthine amine salt. The compound of formula I is KMUP-2 amine salt, wherein R₂ is methoxy group and R₄ is hydrogen, which has the chemical name 7-[2-[4-(2-methoxybenzene)piperazinyl]ethyl]-1,3-dimethylxanthine amine salt. In another embodiment, the compound of formula I also is KMUP-3 amine salt, wherein R₂ is hydrogen and R₄ is nitro group, which has the chemical name 7-[2-[4-(4-nitrobenzene)piperazinyl]ethyl]-1,3-dimethylxanthine amine salt. In another embodiment, the compound of formula I also is KMUP-4 amine salt, wherein R₂ is nitro group and R₄ is hydrogen, which has the chemical name 7-[2-[4-(2-nitrobenzene)piperazinyl]ethyl]-1,3-dimethylxanthine amine salt.

To achieve the above purpose, formula I salts can be synthetically produced from the 2-Chloroethyltheophylline compound and piperazine substituted compound.

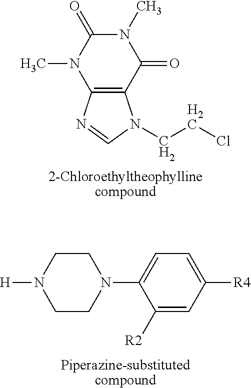

The compounds of formula I salts set forth in the examples below were prepared using the following general procedures as indicated.

The general procedure 1 includes steps of dissolving 2-Chloroethyl theophylline and piperazin substituted compound in hydrous ethanol solution, and the amount of reagent should be conjugated depending on the molecular weight percentage. After adding the strong base e.g. sodium hydroxide (NaOH) or sodium hydrogen carbonate (NaHCO₃) to make the solution more alkaline or more basic, a heating procedure is performed under reflux for three hours. Allowed to stand overnight, the cold supernatant was decanted for proceeding, efficient removal of solvents by vacuum concentration, and then the residue were dissolved with one-fold volume of ethanol and three-fold volume of 2N hydrochloric acid (HCl), kept at 50° C. to 60° C. to make a saturated solution (pH 1.2). The saturated solution was sequentially treated, decolorized with activated charcoal, filtered, deposited overnight and filtered to obtain KMUP-1 HCl with a white crystal.

The general procedure 2 includes steps of dissolving 2-Chloroethyl theophylline and piperazine-substituted compounds in hydrous ethanol solution, and the amount of reagent should be conjugated depending on the molecular weight percentage. Then, a heating procedure is performed under reflux for three hours. Allowed to stand overnight, the cold supernatant was decanted for proceeding, efficient removal of solvents by vacuum concentration, and then the residue were dissolved with one-fold volume of ethanol and three-fold volume of 2N hydrochloric acid (HCl), kept at 50° C. to 60° C. to make a saturated solution (pH 1.2). The saturated solution was sequentially treated, decolorized with activated charcoal, filtered, deposited overnight and filtered to obtain KMUP-1 HCl with a white crystal.

According to the general procedure 1 or 2, KMUPs amine salts compound of formula I can be synthetically produced directly, from the 2-Chloroethyltheophylline compound, piperazine substituted compound and carboxylic acid selected from the group of RX. Thereby, KMUPs compound may represent KMUPs amine salts. Preferably, in one embodiment, KMUP-1 is dissolved in a mixture of ethanol and γ-Polyglutamic acid. The solution is reacted at warmer temperature, the methanol is added thereinto under room temperature, and the solution is incubated over night for crystallization and filtrated to obtain KMUP-1-γ-Polyglutamic acid salt.

According to the above-mentioned aspect of the present invention, KMUPs quaternary amine salt compounds are preferred to inhibit monocrotaline (MCT)-induced proliferation of pulmonary artery and pulmonary artery hypertension. Specifically speaking, KMUPs amine salt compounds in one embodiment, as KMUP-1-Atorvastatin salt, KMUP-2-Atorvastatin salt, KMUP-3-Atorvastatin salt, KMUP-4-Atorvastatin salt; KMUP-1-Cerivastatin salt, KMUP-2-Cerivastatin salt, KMUP-3-Cerivastatin salt, KMUP-4-Cerivastatin salt; KMUP-1-Fluvastatin salt, KMUP-2-Fluvastatin salt, KMUP-3-Fluvastatin salt, KMUP-4-Fluvastatin salt; KMUP-1-Lovastatin salt, KMUP-2-Lovastatin salt, KMUP-3-Lovastatin salt, KMUP-4-Lovastatin salt; KMUP-1-Mevastatin salt, KMUP-2-Mevastatin salt, KMUP-3-Mevastatin salt, KMUP-4-Mevastatin salt; KMUP-1-Pravastatin salt, KMUP-2-Pravastatin salt, KMUP-3-Pravastatin salt, KMUP-4-Pravastatin salt; KMUP-1-Rosuvastatin salt, KMUP-2-Rosuvastatin salt, KMUP-3-Rosuvastatin salt, KMUP-4-Rosuvastatin salt; KMUP-1-Simvastatin salt, KMUP-2-Simvastatin salt, KMUP-3-Simvastatin salt, KMUP-4-Simvastatin salt; KMUP-1-CMC salt, KMUP-2-CMC salt, KMUP-3-CMC salt, KMUP-4-CMC salt; KMUP-1-hyaluronic acid salt, KMUP-2-hyaluronic acid salt, KMUP-3-hyaluronic acid salt, KMUP-4-hyaluronic acid salt; KMUP-1-polyacrylic acid salt, KMUP-2-polyacrylic acid salt, KMUP-3-polyacrylic acid salt, KMUP-4-polyacrylic acid salt; KMUP-1-Eudragit salt, KMUP-2-Eudragit salt, KMUP-3-Eudragit salt, KMUP-4-Eudragit salt; KMUP-1-polylactide salt, KMUP-2-polylactide salt, KMUP-3-polylactide salt, KMUP-4-polylactide salt; KMUP-1-polyglycolic acid salt, KMUP-2-polyglycolic acid salt, KMUP-3-polyglycolic acid salt, KMUP-4-polyglycolic acid salt; KMUP-1-dextran sulfate salt, KMUP-2-dextran sulfate salt, KMUP-3-dextran sulfate salt, KMUP-4-dextran sulfate salt; KMUP-1-heparan sulfate salt, KMUP-2-heparan sulfate salt, KMUP-3-heparan sulfate salt, KMUP-4-heparan sulfate salt; KMUP-1-alginate salt, KMUP-2-alginate salt, KMUP-3-alginate salt, KMUP-4-alginate salt; KMUP-1-γ-PGA salt, KMUP-2-γ-PGA salt, KMUP-3-γ-PGA salt, KMUP-4-γ-PGA salt; KMUP-1-APA salt, KMUP-2-APA salt, KMUP-3-APA salt, KMUP-4-APA salt etc.

In accordance with a further aspect of the present invention, depending on the desired clinical use and the effect, the adaptable administration method of KMUPs pharmaceutical composition includes one selected from a group consisting of an oral administration, an intravenous injection, a subcutaneous injection, an intraperitoneal injection, an intramuscular injection and a sublingual administration.

The term excipients or “pharmaceutically acceptable carrier or excipients” and “bio-available carriers or excipients” mentioned above include any appropriate compounds known to be used for preparing the dosage form, such as the solvent, the dispersing agent, the coating, the anti-bacterial or anti-fungal agent and the preserving agent or the delayed absorbent. Usually, such kind of carrier or excipient does not have the therapeutic activity itself. Each formulation prepared by combining the derivatives disclosed in the present invention and the pharmaceutically acceptable carriers or excipients will not cause the undesired effect, allergy or other inappropriate effects while being administered to an animal or human. Accordingly, the derivatives disclosed in the present invention in combination with the pharmaceutically acceptable carrier or excipients are adaptable in the clinical usage and in the human. A therapeutic effect can be achieved by using the dosage form in the present invention by the local or sublingual administration via the venous, oral, and inhalation routes or via the nasal, rectal and vaginal routes. About 0.1 mg to 1000 mg per day of the active ingredient is administered for the patients of various diseases.

The carrier is varied with each formulation, and the sterile injection composition can be dissolved or suspended in the non-toxic intravenous injection diluents or solvent such as 1,3-butanediol. Among these carriers, the acceptable carrier may be mannitol or water. Besides, the fixing oil or the synthetic glycerol ester or di-glycerol ester is the commonly used solvent. The fatty acid such as the oleic acid, the olive oil or the castor oil and the glycerol ester derivatives thereof, especially the oxy-acetylated type, may serve as the oil for preparing the injection and as the naturally pharmaceutical acceptable oil. Such oil solution or suspension may include the long chain alcohol diluents or the dispersing agent, the carboxylate methyl cellulose (CMC) or the analogous dispersing agents, such as methyl cellulose, ethyl cellulose and hydroxyl ethyl methyl cellulose (HEMC). Other carriers are common surfactant such as Tween and Spans or other analogous emulsion, or the pharmaceutically acceptable solid, liquid or other bio-avaliable enhancing agent used for developing the formulation that is used in the pharmaceutical industry.

The composition for oral administration adopts any oral acceptable formulation, which includes capsule, tablet, pill, emulsion, aqueous suspension, dispersing agent and solvent. The carrier is generally used in the oral formulation. Taking the tablet as an example, the carrier may be the lactose, the corn starch and the lubricant, and the magnesium stearate is the basic additive. The diluents used in the capsule include the lactose and the dried corn starch. For preparing the aqueous suspension or the emulsion formulation, the active ingredient is suspended or dissolved in an oil interface in combination with the emulsion or the suspending agent, and the appropriate amount of the sweetening agent, the flavors or the pigment is added as needed.

The nasal aerosol or inhalation composition may be prepared according to the well-known preparation techniques. For example, the bioavailability can be increased by dissolving the composition in the phosphate buffer saline and adding the benzyl alcohol or other appropriate preservative, or the absorption enhancing agent. The compound of the present invention may be formulated as suppositories for rectal or virginal administration.

The compound of the present invention can also be administered intravenously, as well as subcutaneously, parentally, muscular, or by the intra-articular, intracranial, intra-articular fluid and intra-spinal injections, the aortic injection, the sterna injection, the intra-lesion injection or other appropriate administrations.

Rats treated with vehicle once a day for 21 days after a single intra-peritoneal injection of monocrotaline (MCT) developed the pulmonary artery hypertension (PAH). Long-term daily treatment with KMUPs for 21 days significantly reduced MCT-induced increases in mean pulmonary arterial pressure (MPAP) as shown in Table 2.

TABLE 2

| | |
|---|---|
| KMUP-1 HCl | 35.8 ± 3.2 |
| KMUP-1-hyaluronic Acid | 12 ± 2.8 |
| KMUP-1-γ-Polyglutamic acid salt | 11 ± 1.9 |
| KMUP-1-CMC | 13 ± 3.5 |
| KMUP-2-polylactic acid | 15 ± 2.6 |
| KMUP-1-Eudragit | 11 ± 3.4 |
| KMUP-3-heparan sulfate | 11 ± 2.6 |
| KMUP-3-γ-Polyglutamic acid | 14 ± 2.8 |
| KMUP-4-CMC | 35.8 ± 3.2 |
| KMUP-1-Atorvastatin | 12 ± 2.8 |
| KMUP-1-alginic acid | 11 ± 1.9 |

P < 0.05; significantly different from control (n = 5)

Plasma 5-HT Levels in MCT-Treated Rats

The plasma concentrations of 5-HT are shown in FIG. 1. The plasma concentration of 5-HT was significantly greater in monocrotaline-induced pulmonary artery hypertension (MCT-PAH) rats than control rats (4.2±0.7 and 3.2±0.3 ng/mL, respectively; P<0.05). The plasma concentration of 5-HT in KMUP-1-treated rats was significantly decreased, compared to non-treated MCT-PAH rats (3.2±0.5 ng/mL, p.o. and 3.2±0.6 ng/mL, i.p. 1 mg/kg, respectively; P<0.05) (FIG. 1).

Effects on 5-HT-Constricted Pulmonary Artery (PA)

5-HT (10 μM) produced a contractile response in pulmonary artery of control group, which was inhibited by KMUP-1 (0.1-100 μM) and simvastatin (0.1-100 μM). In the presence of the NOS inhibitor L-NAME (100 KMUP-1 (10-100 μM) reduced the maximum response of pulmonary artery to 5-HT (P<0.05). L-NAME itself did not induce any constriction, but it enhanced 5-HT-induced constriction. KMUP-1 reversed the 5-HT-induced constriction in rat pulmonary artery in a concentration-dependent manner. L-NAME did not significantly reduce this reversal (FIG. 2).

Intracellular Calcium Response to 5-HT

5-HT (10 μM) induced a calcium influx in pulmonary arterial smooth muscle cells (PASMCs). In the following set of experiments, focused whether KMUP-1 inhibited 5-HT-induced calcium influx (FIG. 3). Δ[Ca²⁺]i indicates the difference in [Ca²⁺]i between basal and peak treated levels induced by 5-HT (FIG. 4). KMUP-1 significantly inhibited the sustained [Ca²]i response to 5-HT by 65% at 10 μM.

Effect of KMUP-1 on PASMC Proliferation in MCT-Treated Rats

In MCT-treated rats administered with vehicle, PCNA labeling indicated the proliferation of PASMCs in distal pulmonary artery walls, which were more marked in MCT-treated groups. The number of PCNA-positive cells (stained brown) was markedly lower in the pulmonary artery walls of rats treated with KMUP-1 (FIG. 5).

Pulmonary 5-HTT Expression and Vascular Immunochemistry

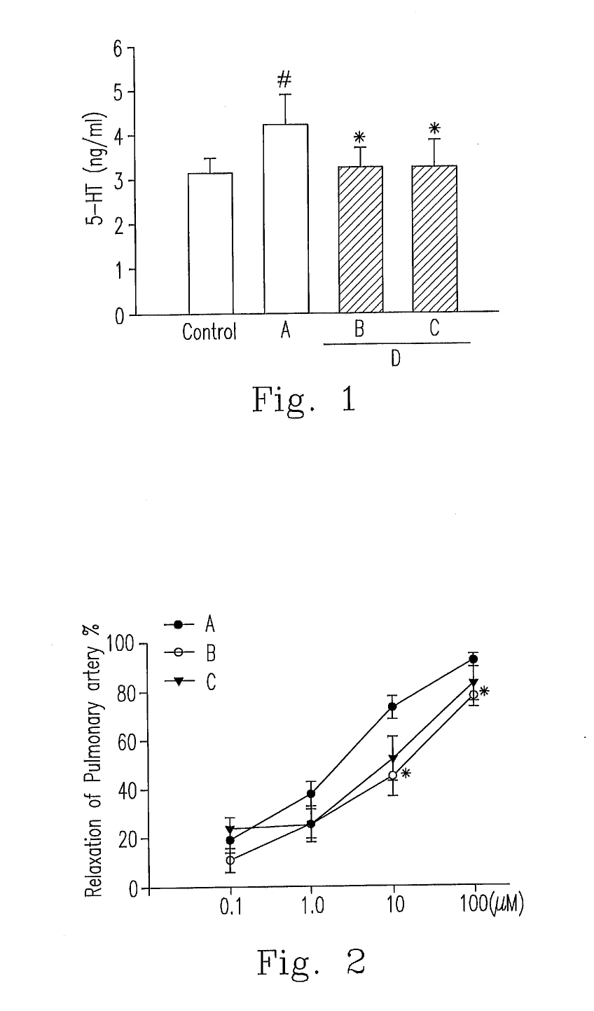

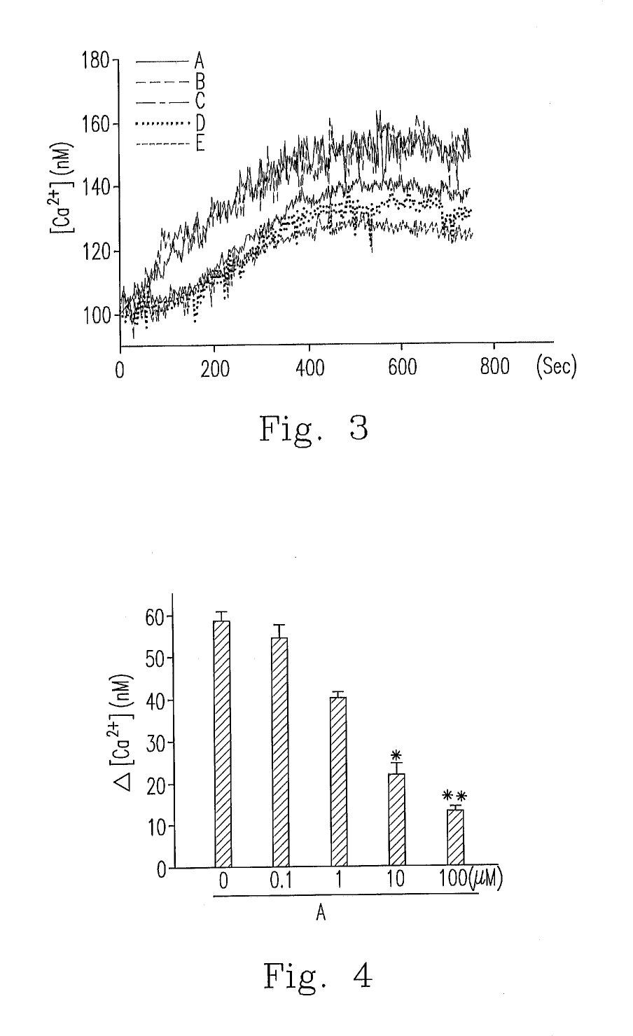

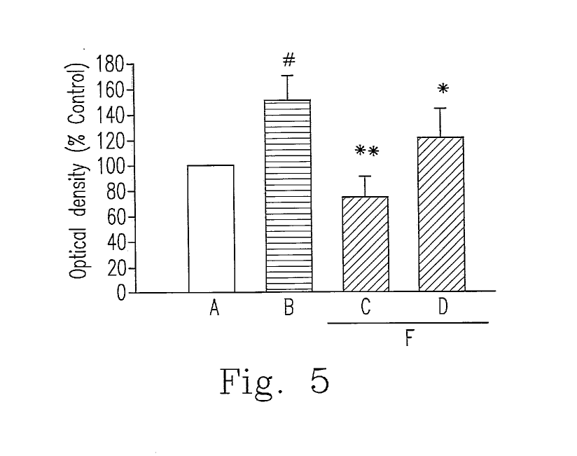

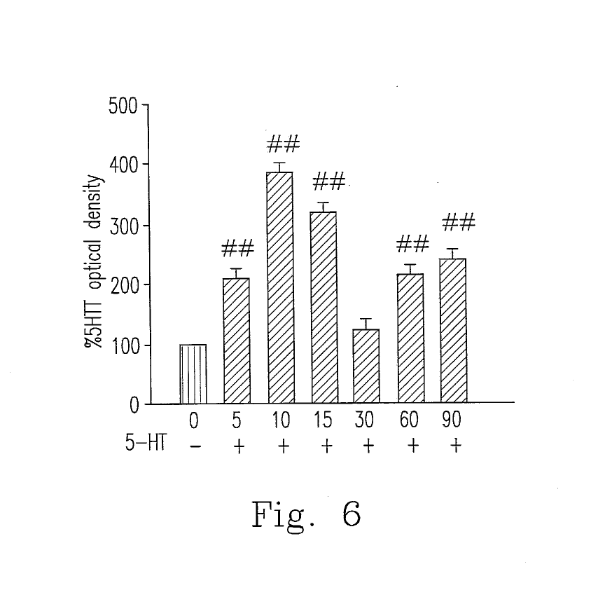

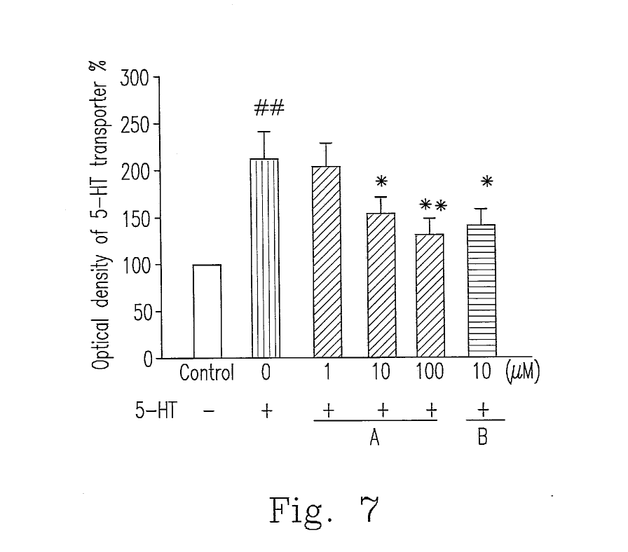

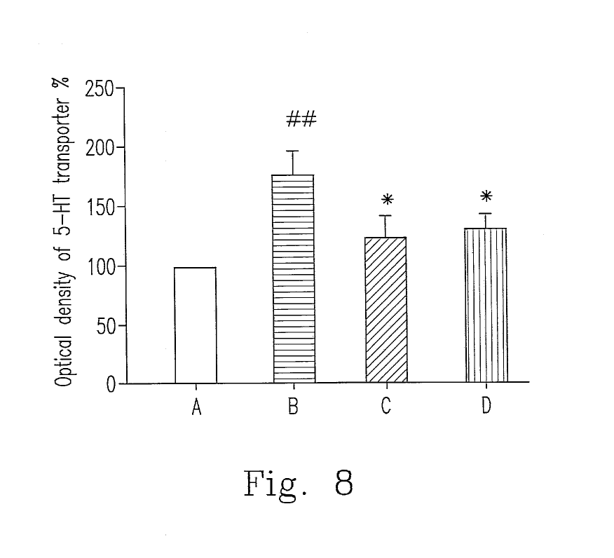

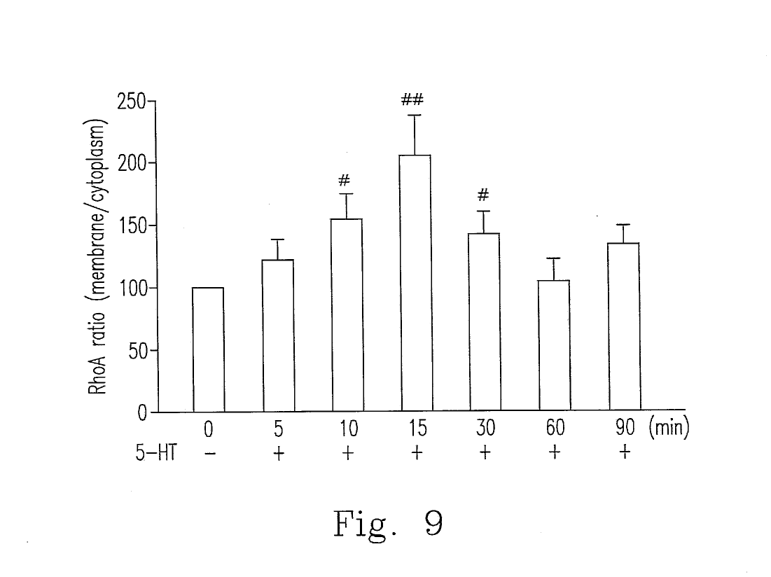

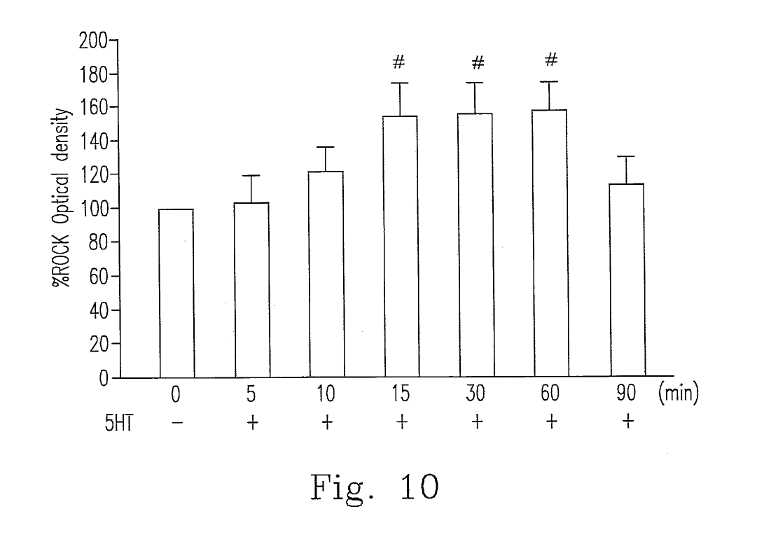

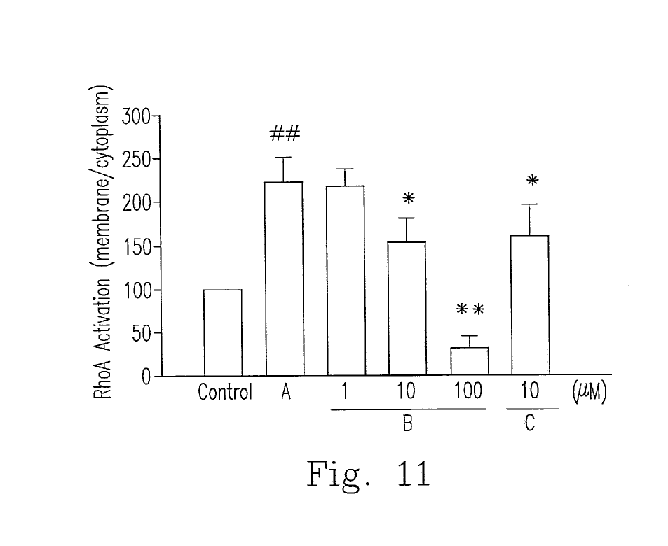

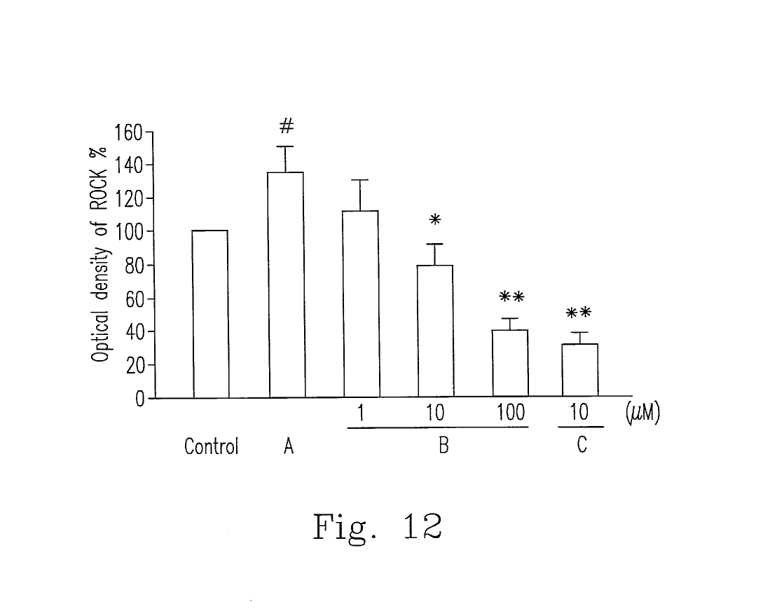

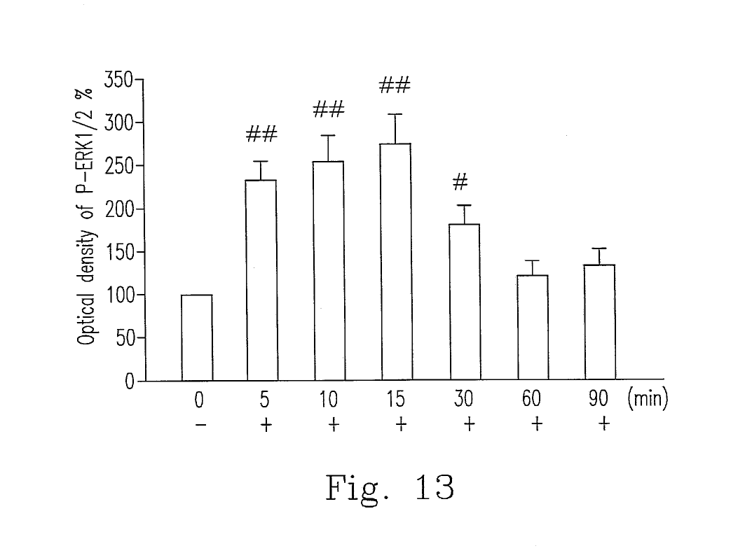

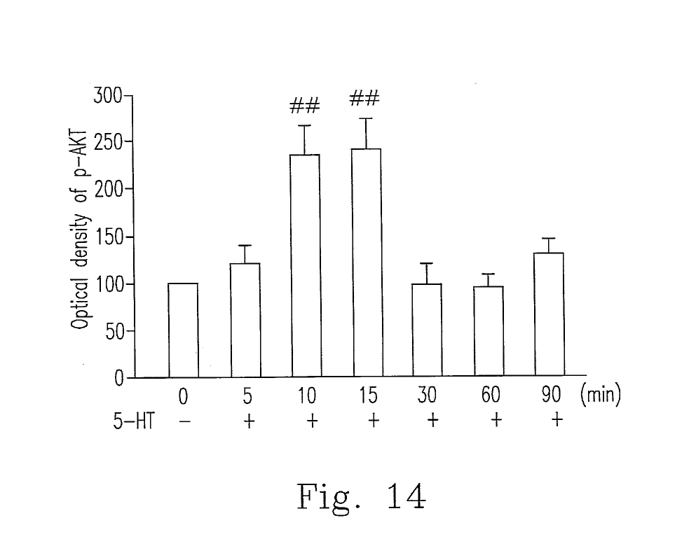

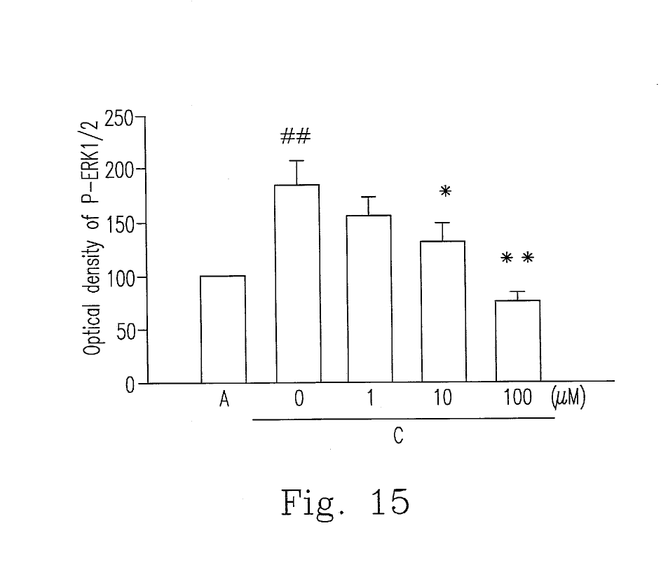

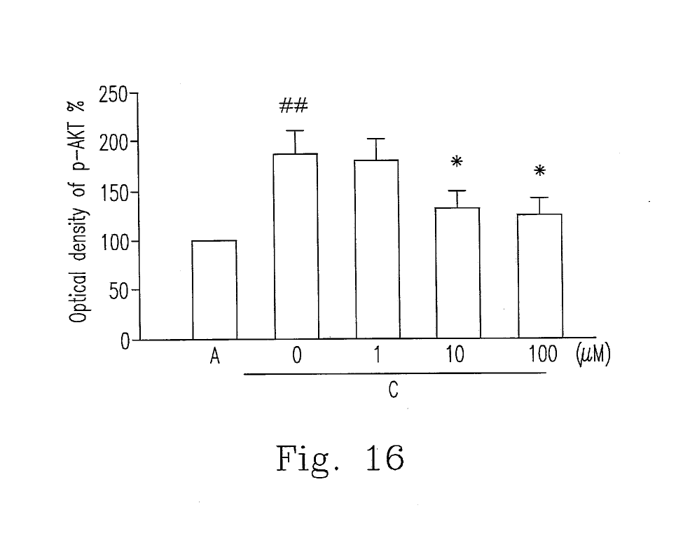

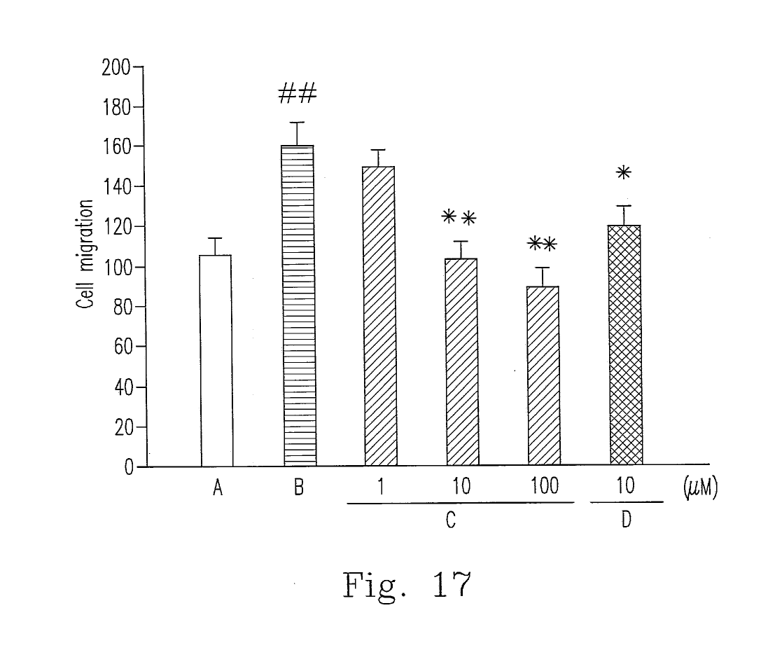

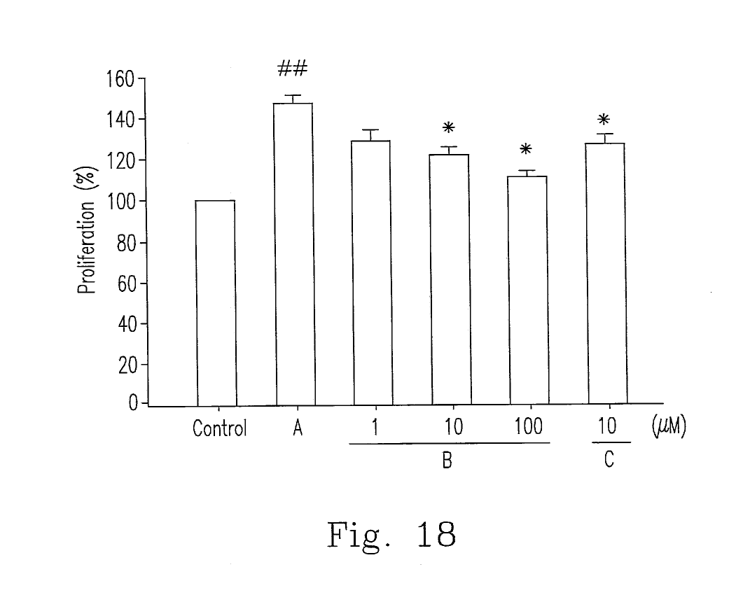

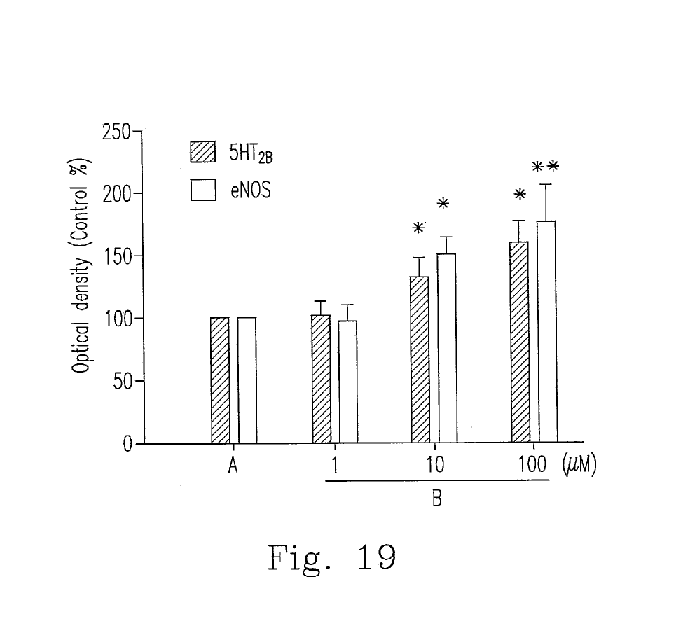

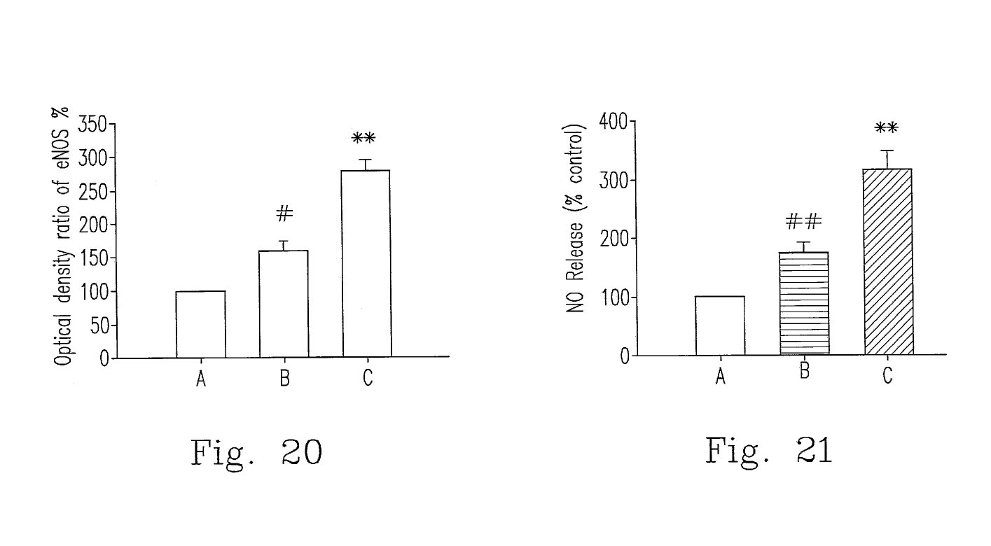

5-HTT expression in pulmonary artery 21 days after MCT injection was significantly decreased after treatment with KMUP-1 at doses of 5 mg/kg p.o. and 1 mg/kg i.p., as assayed by immunochemistry (FIG. 6). Western blotting measurement of 5-HTT protein expression in lung showed similar results. 5-HTT protein expression was significantly reduced by administration of KMUP-1 in lung tissue (FIG. 7).

5-HTT Expression in PASMCs

PASMCs were treated with 5-HT (10 μM) for 5-90 min. We found that 5-HTT protein expression achieved a peak 10 min after 5-HT treatment (FIG. 8). Cells were first treated with KMUP-1 (1-100 μM) and Y27632 (10 μM) for 24 h and then with 5-HT for 10 min. Y27632 (10 μM) and KMUP-1 dose-dependently inhibited 5-HT-induced expression of 5-HTT (FIG. 9). KMUP-1 and simvastatin at 10 μM also both inhibited 5-HT-induced 5-HTT expression (FIG. 10).

RhoA Translocation and ROCK Expression in PASMCs

To test whether RhoA activation was involved in 5-HT-treated PASMCs, we evaluated RhoA activity by measuring the membrane-tocytosol ratio of RhoA expression. 5-HT (10 μM) stimulated an increase in membrane/cytosol RhoA in PASMCs at 5 min, with a peak at 15 min and a return to basal levels by 60 min (FIG. 11). ROCK expression induced by 5-HT was significantly increased at 15-60 min and then returned to basal levels by 90 min (FIG. 12).

Attenuation of 5-HT-Induced RhoA Membrane Localization and ROCK

Since 5-HT stimulation led to increased levels of activated RhoA in PASMCs, and KMUP-1 treatment at 1-100 μM inhibited 5-HT stimulated RhoA translocation (FIG. 13), examined the effect of KMUP-1 on 5-HT-induced ROCK. Treatment with KMUP-1 (1-100 μM) significantly inhibited 5-HT-induced ROCK (FIG. 14). Activation of RhoA is associated with its translocation from the cytosol to the membrane. Stimulation of PASMCs with 5-HT (10 μM, 15 min) led to an increased level of membrane-associated RhoA. 24 h treatment with KMUP-1 dose-dependently reversed the 5-HT-induced RhoA membrane association. Y27632 (10 μM) also inhibited the membrane-to-cytosol ratio of RhoA and ROCK expression.

Attenuation of 5-HT-Induced Activation of ERK1/2 and AKT

Phosphorylation or activation of ERK1/2 and AKT kinase is associated with proliferation in a variety of cell types, including PASMCs. Exposure of cells to 5-HT (10 μM) for 15 min (peak time for ERK1/2 and AKT phosphorylation) triggered 2.3-fold increases phosphorylation of ERK1/2 (FIG. 15) and AKT (FIG. 16). This response was dose-dependently inhibited by pre-incubation of cells with KMUP-1 (1-100 μM) for 24 h (FIG. 17, 18).

Inhibition of 5-HT-Induced PASMCs Migration and Proliferation

Next investigated 5-HT-induced pulmonary arterial smooth muscle cells (PASMCs) migration using a wound assay model. Stimulation of cells with 5-HT (10 μM) for 24 h enhanced cell migration in a wound healing assay. Treatment with KMUP-1 (10-100 μM) and simvastatin (10 μM) inhibited 5-HT induced PASMCs migration (FIG. 19). PASMCs proliferation, tested by MTT assay, was also dose-dependently inhibited by KMUP-1 and simvastatin (10 μM) (FIG. 20).

TABLE 1
Estimated IC₅₀ and Kᵢ values of KMUP-1 in
radioligand binding to 5-HT_{2A}, 5-HT_{2B}, and 5-HT_{2C}
receptors in human recombinant CHO-K1 cells.

| | radioligand | IC₅₀ [μM] | Kᵢ values [μM] |
|---|---|---|---|
| 5-HT_{2A} | [³H]-ketanserin | 0.34 | 0.0971 |
| 5-HT_{2B} | [³H]-LSD | 0.04 | 0.0254 |
| 5-HT_{2C} | [³H]-mesulergine | 0.408 | 0.214 |

Radioligand Binding on 5-HT_{2A}, 5-HT_{2B} and 5-HT_{2C} Receptors

As shown in Table 1, the ligand specificity of the human 5-HT_{2B} receptor to KMUP-1 was more selective than the 5-HT_{2A} and 5-HT_{2C} receptors. The affinity of these sub-receptors for KMUP-1 is 5-HT_{2A}>5-HT_{2B}>5-HT_{2C}.

The Expression of eNOS, 5-HT_{2B} Receptor and the Production of NO in Human Pulmonary Artery Endothelial Cell (HPAEC)

eNOS and 5-HT_{2B} receptor expressions were assessed by Western blotting. Incubation of HPAEC with KMUP-1 (1-100 μM) for 24 h dose-dependently increased eNOS and 5-HT_{2B} receptor expressions in cultured HPAEC (FIG. 9). Incubation of 5-HT (10 μM) for 30 min in HPAEC, 5-HT increased the expression of eNOS and the release of NO, compared to the control group. Treatment of HPAEC with KMUP-1 (10 μM) for 24 h before adding 5-HT also raised the expression of eNOS and the release of NO, compared to 5-HT treatment alone (FIGS. 10A and B).

Animal Models and Hemodynamic Measurement

All experiments were performed in adult male Wistar rats (300 to 350 g) in accordance with institutional guidelines after approval by the ethical review committee. PAH development and pulmonary expression of 5-HTT were examined in rats after single injection of MCT (60 mg/kg i.p.). To assess the potential preventive effect of KMUP-1 on MCT-induced PAH and associated proliferation, we assigned rats at random to 2 groups of 8 animals which received KMUP-1 at 5 mg/kg/day p.o or 1 mg/kg/day i.p. All treatments were given once a day for 3 weeks after a single MCT injection (60 mg/kg i.p.) (Abe et al., 2004). On day 21, rats were anesthetized and pulmonary artery blood pressure (MPAP) was recorded as previously described (Chung et al., 2010). Lung tissues were dissected for Western blotting and immunohistochemistry.

Reagents and Antibodies

KMUPs was synthesized in our laboratory and dissolved in distilled water. All other reagents were from Sigma (St. Louis. Mo, USA) unless otherwise specified. Anti-RhoA monoclonal antibody and anti-ERK1/2 rabbit antibody were purchased from Santa Cruz Biotechnology (CA, USA). Anti-5-HT_{2B}, anti-eNOS and anti-ROCK (ROCKII) antibody were purchased from Upstate Biotechnology (Lake Placid, N.Y., USA). Anti-5-HTT rabbit antibody was purchased from Chemicon Biotechnology (Temecula, Calif., USA). Anti-phosphor-ERK1/2, anti-AKT, anti-phospho-AKT, and horseradish peroxidase-conjugated polyclonal rabbit and mouse antibody were purchased from Santa Cruz Biotechnology. [³H]mesulergine was purchased from Amersham (Buckinghamshire, UK). [³H]ketanserin was purchased from Perkin-Elmer (Shelton, Conn., USA).

Measurement of Plasma 5-HT Levels

After MCT-treatment, rats acquired severe PAH and received sodium pentobarbital (40 mg/kg, i.p.) at day 21 for surgical anesthesia and measurement of MPAP (Chung et al., 2010). Blood samples (1.0 mL) were obtained from the heart. Blood was transferred to plastic tubes that included EDTA and was centrifuged at 100 g for 20 min. The plasma was transferred to tubes and stored at −80° C. until analysis. Plasma 5-HT levels were determined using commercially available Serotonin EIA (IBL, Minneapolis, USA). Cross-reactivity with related substances (e.g., 5-HIAA, phenylalanine, histidine and tyramine) has been reported to be <0.002%. Results were read from a standard curve. The threshold of detection was 0.3 ng/mL.

Isometric Force of Pulmonary Arterys (PAs)

Wistar rats were euthanized with an overdose of sodium pentobarbital (60 mg/kg, i.p.) before open-chest surgery. During surgery, a thoracic retractor was used to help isolate the pulmonary artery. The chest was opened to dissect the second branches of the main pulmonary artery, which were cut into 2-3 mm rings, suspended under isometric conditions and connected to a force transducer as previously described (Ugo Basile, Model 7004, Comerio-VA, Italy) (Schach et al., 2007) to measure the constriction caused by 5-HT (10 μM). The pulmonary artery ring preparations were stretched to a basal tension of 1 g and allowed to equilibrate for 60-90 min. After equilibration, pulmonary artery rings were constricted with 5-HT (10 μM) to prime the tissues and check the functionality of the endothelium (at least 80% relaxation in response to acetylcholine 1 μM). Once the contractile response to each agonist reached a stable tension, KMUP-1 (0.1-100 μM) was cumulatively added to the organ bath in the presence of 5-HT (10 μM, pre-incubation time of 15 min).

The effect of KMUP-1 on NOS and 5-HT was also studied in vessels pre-incubated with the NOS inhibitor L-NAME (100 μM) 20 min before 5-HT administration. The percentage of relaxation was estimated using the following equation: relaxation (%)=(maximal contraction—relaxation level)/(maximal contraction-basal level)×100. Data was obtained from serotonin-induced maximal contractile responses in pulmonary artery.

PASMCs Proliferation in MCT-Treated Lung Tissues

Evaluated proliferating cell nuclear antigen (PCNA) to assess PASMCs proliferation in rats treated with MCT alone or with KMUP-1. Lung tissue sections were de-paraffinized in xylene and then treated with a graded series of alcohol washes, rehydrated in PBS (pH 7.5), and incubated with target retrieval solution (DAKO Co, Tokyo, Japan) in a water bath at 90° C. for 20 min. Endogenous peroxidase activity was blocked with H₂O₂ in PBS (3%, vol/vol) for 5 min. Slides were then washed in PBS, incubated for 30 min in a protein blocking solution, and incubated for 30 min with anti-PCNA mouse monoclonal antibody (PC-10, 1:200, Dako). Antibodies were washed off, and the slides were processed with an alkaline phosphatase LSAB+system horseradish peroxidase detection kit (DAKO Co, Tokyo, Japan). Brown color was generated with a DAB substrate, and nuclei were counter-stained with hematoxylin.

Lung 5-HTT Immunohistochemical Analysis

For 5-HTT immunostaining, the lung slides were dewaxed in 100% xylene, and the sections were then rehydrated by successive immersion first in decreasing ethanol concentrations (100%, 90%, 80%, 50% and 30%) and then in water. Endogenous peroxidase activity was blocked using H₂O₂ in methanol (0.3% vol/vol) for 10 min. After three PBS washes, sections were pre-incubated in PBS supplemented with 3% (wt/vol) BSA for 30 min, then incubated overnight at 4° C. with goat polyclonal anti-5-HTT antibody (Abcam Biotechnology, Cambridge, UK) diluted to 1:1000 in 1×PBS, 0.02% BSA. Next, the sections were exposed for 1 h to biotin-labeled anti-goat secondary antibodies (DAKO Co, Tokyo, Japan) diluted 1:1000 in the same buffer. Peroxidase staining of the slides incubated in streptavidin-biotin horseradish peroxidase solution was carried out using 3,3′-diaminobenzidine tetrahydrochloride dihydrate (DAB; DAKO Co, Tokyo, Japan) and hydrogen peroxide. Finally, the sections were stained with hematoxylin and eosin.

Preparation of Pulmonary Arterial Smooth Muscle Cells (PASMCs)

Wistar rats were anesthetized with an overdose of sodium pentobarbital (60 mg/kg) and the skin was sterilized with 75% alcohol. The chest was opened and the heart and lung were removed. The organs were rinsed several times in PBS. The pulmonary arterys (PAs) were segregated in a sterile manner. The outer sphere was peeled and the microtubule was snipped visually, and endothecia were shaved lightly 2-3 times in order to remove endothelial cells. The tunica media was prepared into scraps (1 mm³) in DMEM (Dulbecco's modified Eagle's medium). PASMCs were cultured in DMEM containing 10% fetal bovine serum (5% CO₂ at 37° C.). The culture medium was changed every 3 days and cells were subcultured until confluence. Primary cultures of 2-4 passages were used in the experiments. Cells were examined by immunofluorescence staining of α-actin to confirm the purity of PASMCs. Over 95% of the cell preparations were found to be composed of smooth muscle cells.

Human Pulmonary Artery Endothelial Cell (HPAEC) Cultured

HPAEC purchased from ATCC were maintained in humidified incubator containing 5% CO₂ at 37° C. HPAEC were cultured in F-12 supplemented with 10% fetal bovine serum. The culture medium was changed every 3 days and cells were subcultured until confluence. HPAEC were harvested with a solution of trypsin-EDTA (GIBCO BRL, NY, USA) while in a logarithmic phase of growth. HPAEC were used between passages 4-8.

Microculture Tetrazolium Test (MTT)

PASMCs were seeded into 96-well plates at a density of 1×10⁴ cells/well. The cells were then incubated in medium containing vehicle (1% FBS DMEM) and 5-HT (10 μmol/L) for 24 h with or without KMUP-1 (1-100 μM) and simvastatin (10 μM) added 30 min before 5-HT. At the end of this period, MTT (2 g/L) was added to each well, and incubation proceeded at 37° C. for 4 h. Thereafter, the medium was removed and the cells were solubilized in 150 μL DMSO. The optical density (OD) of each well was determined by enzyme-linked ELISA at 540 nm of wavelength.

PASMCs [Ca²⁺]i Measurement

The measurement of [Ca²⁺]i in PASMCs was performed using a spectrofluorophotometer as previously reported (Wang et al., 2004). PASMCs, cultured for 2-4 passages and re-suspended by trypsin, were loaded with Fura-2/AM for the measurement of [Ca²⁺]i changes in cells within the cuvette by spectrofluorophotometer (Shimadzu, RF-5301PC, Shimadzu, Japan). KMUP-1 was applied 5 min before application of 5-HT (10 μM).

Western Blotting

PASMCs were stimulated with 5-HT or not, as indicated. Cells were treated with KMUP-1 for 24 h before 5-HT stimulation. Whole lysates were collected and resolved by SDS-PAGE as previously described (Liu et al., 2004). Primary antibodies were anti-β-actin at: 1:10,000 dilution and anti-RhoA, anti-ROCK, antiphospho-ERK1/2, anti-ERK1/2, anti-phospho-AKT, anti-AKT, anti-5HTT, anti-5-HT_{2B} and anti-eNOS at 1:1000. All blots were incubated with antibodies at 4° C. overnight. After being washed, the appropriate secondary antibodies were added at a dilution of 1:1000 for 1 h at room temperature. After extensive washing, blots were developed with a Super Signal enhanced chemiluminescence kit (Biorad, CA, USA) and visualized on Kodak AR film.

RhoA Translocation

Previous reports have shown that the active form of RhoA is translocated from the cytosol to the plasma membrane, where it activates Rho kinase (Gong et al., 1997)). Therefore, the membrane/cytosol ratio of RhoA is considered a measure of RhoA activity. The cytosol and membrane protein of PASMCs were extracted with a CNM (cytosol, nuclear, membrane) kit. To assess membrane trans-location of RhoA, protein in membrane and cytosolic fractions was determined by standard Western blot analysis using mouse monoclonal anti-RhoA antibody (1:1000 dilution, Santa Cruz Biotechnology) and a peroxidase-labeled anti-mouse immunoglobulin (Ig) G antibody (1:1000 dilution, Santa Cruz Biotechnology, CA, USA). The relative density of membrane to cytosolic RhoA was determined using NIH imaging software.

Expression of 5-HTT and RhoA/ROCK and Phosphorylation of ERK1/2 and AKT Kinase

The expression of RhoA/ROCK and phosphorylation of ERK1/2 and AKT kinase in PASMCs were assessed by incubating the PASMCs with 5-HT (10 μM) for 15 min (peak time for RhoA/ROCK, ERK1/2 and AKT phosphorylation) and 5-HTT for 10 min (peak time for 5-HTT) after KMUP-1 (1-100 μM) was added to the culture of PASMCs for 24 h.

Expression of eNOS and 5-HT_{2B} Receptor in HPAEC

To measure the expression of eNOS and 5-HT_{2B} receptor in HPAEC after incubation with KMUP-1(1-100 μM) for 24 h, whole lysates were collected for Western blotting assay. The relative density was determined using NIH imaging software.

Nitric Oxide Production in Human Pulmonary Artery Endothelial Cell (HPAEC)

Production of NO in HPAEC was determined using the Griess method (Niwano et al., 2003). The three step Greiss test converts nitrate (NO₃⁻) into nitrite (NO₂⁻) giving a total NO₂⁻ concentration from a standard calibration curve. HPAEC were incubated with or without KMUP-1 (10 μM) for 24 h and then incubated with 5-HT (10 μmol/L) for 30 min. Media samples from HPAEC were taken and the NO concentration was determined. Six independent experiments were carried out and data are reported as the mean mean±SEM.

Cell Migration Assay Under Microscope

Migration of PASMCs was assessed using a wound assay model (Kimura et al., 2001) in which cells grown to confluence on 6 well dishes were scraped with the edge of a fine razor. The wound edge was viewed and photographed under a microscope (Nikon, Tokyo, Japan) before and after culture for 24 h in serum-free DMEM in the presence of 5HT (10 μM). The distance the cells migrated from the wound surface was then manually measured.

Displacement of Radioligand Binding Assay

As described previously (Bonhaus et al., 1995), full length clones of 5-HT_{2A}, 5-HT_{2B} and 5-HT_{2C} receptor were prepared from CHO-K1 cells. On the basis of higher affinity with [³H]-radiolabeled ketanserin, [³H]-radiolabeled lysergic acid diethylamide (LSD) and [³H]-radiolabeled mesulergine (0.15-1.2 nM) measured the gene products of human 5-HT_{2A} and 5-HT_{2B} receptor and 5-HT_{2C} receptor; respectively, were used to test KMUP-1's effects on radioligand binding ability. IC₅₀ represents the concentration of competing ligand which displaces 50% of the specific binding of the radioligand. Kᵢ values for competition curves were calculated using the Cheng-Prusoff equation (Cheng, 2001).

Compounds

KMUPs were synthesized in our laboratory (Chung et al., 2010). 5-[2-ethoxy-5-(4-methylpiperazin-1-yl)sulfonylphenyl]-1-methyl-3-propyl-4H-pyrazolo[4,3-d]pyrimidin-7-one (Sildenafil) was supplied by Cadila Healthcare Ltd. (Maninagar, India). (R)-(+)-trans-4-(1-Aminoethyl)-N-(4-Pyridyl)cyclohexane-carboxamide dihydrochloride monohydrate (Y27632), N^{ω}-nitro-L-arginine methyl ester (L-NAME), 2,2-dimethylbutanoic acid (1S,3R,7S,8S,8aR)-1,2,3,7,8,8a-hexahydro-3,7-dimethyl-8-[2-[(2R,4R)-tetra-hydro-4-hydroxy-6-oxo-2H-pyran-2-yl]ethyl]-maphthalenyl ester (simvastatin), 5-hydroxytryptamine (5-HT), (3R,4R,5R,13aR,13bR)-4,5-dihydroxy-3,4,5-trimethyl-4,5,8,10,12,13,13a, and 13b-octahydro-2H-[1,6]dioxacycloundecino[2,3,4-gh]pyrrolizine-2,6(3H)-dione (monocrotaline, MCT) were purchased from Sigma-Aldrich (St. Louis Mo., U.S.A.).

Statistical Evaluation

The results were expressed as means±SE. Statistical differences were determined by independent and paired Student's t-test in unpaired and paired samples, respectively. Whenever a control group was compared with KMUP-1 and other treated groups, one way ANOVA or two way repeated measures ANOVA was used. When the ANOVA manifested a statistical difference, the Dunnett's or Student-Newman-Keuls test was applied. AP value less than 0.05 was considered to be significant in all experiments. Analysis of the data and figure plotting was done with SigmaPlot software (Version 8.0, SPSS Scientific, Illinois, Chicago, Ill., USA) and SigmaStat (Version 2.03, SPSS Scientific, Illinois, Chicago, Ill., USA) run on an IBM-compatible computer.

### EMBODIMENTS

### Example 1

### Preparation of KMUP-1 HCl

2-Chloroethyl theophylline (8.3 g), NaOH (8.3 g) and 2-chlorobenzene-piperazine (8.3 g) are dissolved in hydrous ethanol (100 mL) and then heated under reflux for 3 h. Allowed to stand overnight, the cold supernatant was decanted for processing, efficient removal of solvents by vacuum concentration, and then the residue were dissolved with one-fold volume of ethanol and three-fold volume of 2N hydrochloric acid (HCl), kept at room temperature, to make a saturated solution (pH 1.2). The saturated solution was sequentially treated, decolorized with activated charcoal, filtered, deposited overnight and filtered to obtain KMUP-1 HCl with a white crystal.

### Example 2

### Preparation of KMUP-1-CMC Salt

Method 1: 20 g Sodium carboxyl methylcellulose is suspended in distill water and added with KMUP-1 HCl (16 g) and methanol 100 ml to reflux in a three-neck reactor, equipped with a condenser, for 1 hour. After cooling, obtained precipitate is dissolved in methanol 100 ml and the resulted solution is incubated for crystallization and filtrated to obtain KMUP-1-CMC salt (35.4 g).

Method 2: KMUP-1 HCl (16 g) is dissolved in methanol (100 ml) and added with sodium CMC (20 g) and refluxed in a three-neck reactor, equipped with a condenser, for 1 hour. After cooling, obtained precipitate is filtrated and re-crystallized with methanol 100 ml to have KMUP-1-CMC salt (35.2 g).

### Example 3

### Preparation of KMUP-1-γ-Polyglutamic acid salt

Method 1: Sodium y-polyglutamic acid (20 g) is suspended in distill water and added with KMUP-1 HCl (16 g) dissolved in methanol 100 ml to reflux in a three-neck reactor, equipped with a condenser, for 1 hour. After cooling, obtained precipitate is dissolved in methanol 100 ml and the resulted solution is incubated for crystallization and filtrated to obtain KMUP-1-γ-Polyglutamic acid salt (35.6 g).

Method 2: KMUP-1 HCl (16 g) is dissolved in methanol 100 ml and added with sodium alginic acid 20 g dissolved in methanol 100 ml to reflux in a three-neck reactor, equipped with a condenser, for 1 hour. After cooling, obtained precipitate is filtrated and re-crystallized with methanol 100 ml to have KMUP-1-γ-Polyglutamic acid salt (35.8 g).

### Example 4

### Preparation of KMUP-1-Alginic acid salt

Method 1: Sodium alginic acid (20 g) is suspended in distill water and added with KMUP-1 HCl (16 g) dissolved in and methanol 100 ml to reflux in a three-neck reactor, equipped with a condenser, for 1 hour. After cooling, obtained precipitate is dissolved in methanol 100 ml and the resulted solution is incubated for crystallization and filtrated to obtain KMUP-1-Alginic acid salt (35.4 g).

Method 2: KMUP-1 HCl 16 g is dissolved in methanol 100 ml, added with sodium alginic acid 20 g dissolved in methanol 100 ml to reflux in a three-neck reactor, equipped with a condenser, for 1 hour. After cooling, obtained precipitate is filtrated and re-crystallized with methanol 100 ml to have KMUP-1-Alginic acid salt (35.6 g).

### Example 7

### Preparation of KMUP-2-polyacrylic acid salt

KMUP-2 HCl (8 g) is dissolved in a mixture of methanol (100 mL) and sodium polyacrylic acid (2.5 g). The solution is refluxed in a three-neck reactor, equipped with a condenser, for 1 hour. After cooling, obtained precipitate is re-dissolved in methanol 100 ml and the resulted solution is incubated for crystallization and filtrated to obtain KMUP-2-polyacrylic acid salt (7.4 g).

### Example 8

### Preparation of KMUP-2-γ-Polyglutamic acid salt

KMUP-2 HCl (8 g) is dissolved in a mixture of methanol (100 mL) and sodium y-Polyglutamic acid (2.5 g). The solution is refluxed reflux in a three-neck reactor, equipped with a condenser, for 1 hour. After cooling, obtained precipitate is re-dissolved in methanol 100 ml and the resulted solution is incubated for crystallization and filtrated to obtain KMUP-2-γ-Polyglutamic acid salt (10.3 g).

### Example 9

### Preparation of KMUP-1-dextran sulfate salt

KMUP-1 HCl (8 g) is dissolved in a mixture of methanol (100 mL) and sodium dextran sulfate (3.5 g). The solution is refluxed reflux in a three-neck reactor, equipped with a condenser, for 1 hour. After cooling, obtained precipitate is re-dissolved in methanol 100 ml and the resulted solution is incubated for crystallization and filtrated to obtain KMUP-1-dextran sulfate salt (10.6 g).

### Example 10

### Preparation of KMUP-4-heparan sulfate salt

KMUP-4 HCl (8.3 g) is dissolved in a mixture of methanol (100 mL) and sodium heparan sulfate (8.5 g). The solution is refluxed in a three-neck reactor, equipped with a condenser, for 1 hour. Obtained precipitate is re-dissolved in methanol 100 ml and the resulted solution is incubated for crystallization and filtrated to obtain KMUP-4-heparan sulfate salt (9.2 g).

### Example 11

### Preparation of KMUP-2-hyaluronic acid salt

KMUP-2 HCl (8 g) is dissolved in a mixture of methanol (100 mL) and sodium hyaluronic acid (2.5 g). The solution is refluxed in a three-neck reactor, equipped with a condenser, for 1 hour. After cooling, obtained precipitate is re-dissolved in methanol 100 ml and the resulted solution is incubated for crystallization and filtrated to obtain KMUP-2-hyaluronic acid salt (9.4 g).

### Example 12

### Preparation of the Composition in Tablet

Tablets are prepared using standard mixing and formation techniques as described in U.S. Pat. No. 5,358,941, to Bechard et al., issued Oct. 25, 1994, which is incorporated by reference herein in its entirety.

| | |
|---|---|
| KMUP-1-hyaluronic acid salt | 140 mg |
| Lactose | qs |
| Corn starch | qs |

### EMBODIMENTS

### Embodiment 1

A complex compound comprising a KMUPs amine salt represented by formula I:

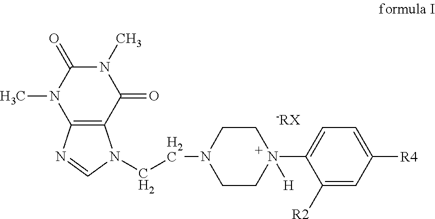

wherein: R2 and R4 are selected independently from a group consisting of a C1˜C5 alkoxy group, a hydrogen, a nitro group, and a halogen atom;
RX includes a carboxylic group selected from a group consisting of a Statin, a sodium carboxyl methylcellulose (sodium CMC), a poly-γ-polyglutamic acid (γ-PGA) derivative and a Co-polymer; and
⁻RX substituent is an anion of the carboxylic group carrying a negative charge, halogen atom is one selected from a group consisting of a fluorine, a chlorine, a bromine and an iodine.

### Embodiment 2

A complex compound for inhibiting MCT-induced pulmonary artery proliferation, comprising a KMUPs amine salt represented by formula I:

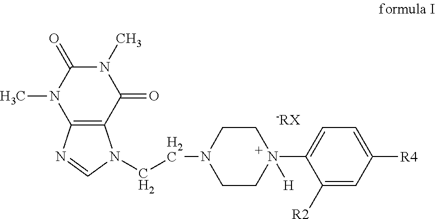

wherein: R2 and R4 are selected independently from a group consisting of a C1˜C5 alkoxy group, a hydrogen, a nitro group, and a halogen atom;
RX includes a carboxylic group selected from a group consisting of a Statin, a sodium carboxyl methylcellulose (sodium CMC), a poly-γ-polyglutamic acid (γ-PGA) derivative and a Co-polymer; and
⁻RX substituent is an anion of the carboxylic group carrying a negative charge, halogen atom is one selected from a group consisting of a fluorine, a chlorine, a bromine and an iodine.

### Embodiment 3

A complex compound for the treatment of 5-HT-induced pulmonary artery hypertension, comprising a KMUPs amine salt represented by formula I:

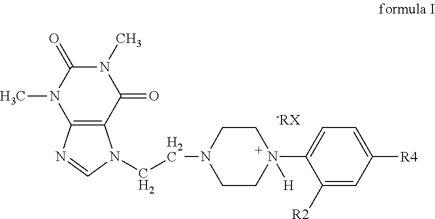

wherein: R2 and R4 are selected independently from a group consisting of a C1˜C5 alkoxy group, a hydrogen, a nitro group, and a halogen atom;
RX includes a carboxylic group selected from a group consisting of a Statin, a sodium carboxyl methylcellulose (sodium CMC), a poly-γ-polyglutamic acid (γ-PGA) derivative and a Co-polymer; and
⁻RX substituent is an anion of the carboxylic group carrying a negative charge, halogen atom is one selected from a group consisting of a fluorine, a chlorine, a bromine and an iodine.

### Embodiment 4

A pharmaceutical composition comprising an effective amount of a KMUPs amine salt represented by formula I:

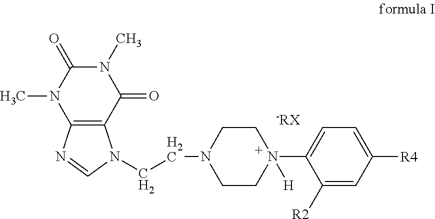

wherein: R2 and R4 are selected independently from a group consisting of a C1˜C5 alkoxy group, a hydrogen, a nitro group, and a halogen atom;
RX includes a carboxylic group selected from a group consisting of a Statin, a sodium carboxyl methylcellulose (sodium CMC), a poly-γ-polyglutamic acid (γ-PGA) derivative and a Co-polymer; and
⁻RX substituent is an anion of the carboxylic group carrying a negative charge, halogen atom is one selected from a group consisting of a fluorine, a chlorine, a bromine and an iodine; and a pharmaceutically acceptable carrier.

### Embodiment 5

A pharmaceutical composition for inhibiting MCT-induced pulmonary artery proliferation, comprising an effective amount of a KMUPs amine salt represented by formula I:

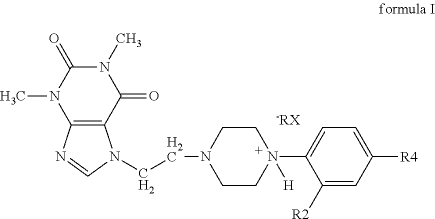

wherein: R2 and R4 are selected independently from a group consisting of a C1˜C5 alkoxy group, a hydrogen, a nitro group, and a halogen atom;
RX includes a carboxylic group selected from a group consisting of a Statin, a sodium carboxyl methylcellulose (sodium CMC), a poly-γ-polyglutamic acid (γ-PGA) derivative and a Co-polymer; and
⁻Rx substituent is an anion of the carboxylic group carrying a negative charge, halogen atom is one selected from a group consisting of a fluorine, a chlorine, a bromine and an iodine; and a pharmaceutically acceptable carrier.

### Embodiment 6

A pharmaceutical composition for treatment of 5-HT-induced pulmonary artery hypertension, comprising an effective amount of a KMUPs amine salt represented by formula I:

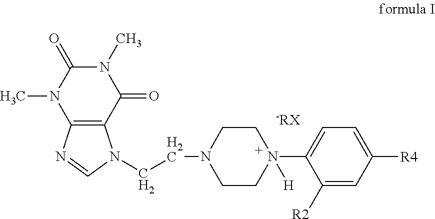

wherein: R2 and R4 are selected independently from a group consisting of a C1˜C5 alkoxy group, a hydrogen, a nitro group, and a halogen atom;
RX includes a carboxylic group selected from a group consisting of a Statin, a sodium carboxyl methylcellulose (sodium CMC), a poly-γ-polyglutamic acid (γ-PGA) derivative and a Co-polymer; and
⁻RX substituent is an anion of the carboxylic group carrying a negative charge, halogen atom is one selected from a group consisting of a fluorine, a chlorine, a bromine and an iodine; and a pharmaceutically acceptable carrier.

### Embodiment 7

A complex compound of any one of Embodiments 1-3, wherein the halogen atom is one selected from a group consisting of a fluorine, a chlorine, a bromine and an iodine.

### Embodiment 8

A complex compound of any one of Embodiments 1-3, wherein the KMUPs amine salt comprising one selected from a group consisting of a KMUP-1-amine salt, a KMUP-2-amine salt, a KMUP-3-amine salt and a KMUP-4-amine salt.

### Embodiment 9

A complex compound of any one of Embodiments 1-3, wherein the KMUP-1-amine salt includes a 7-[2-[4-(2-chlorophenyl)piperazinyl]ethyl]-1,3-dimethylxanthine-amine salt.

### Embodiment 10

A complex compound of any one of Embodiments 1-3, wherein the KMUP-2-amine salt includes a 7-[2-[4-(2-methoxybenzene)piperazinyl]ethyl]-1,3-dimethylxanthine-amine salt.

### Embodiment 11

A complex compound of any one of Embodiments 1-3, wherein the KMUP-3-amine salt includes a 7-[2-[4-(2-nitrobenzene)piperazinyl]ethyl]-1,3-dimethylxanthine-amine salt.

### Embodiment 12

A complex compound of any one of Embodiments 1-3, wherein the KMUP-3-amine salt includes a 7-[2-[4-(2-nitrobenzene)piperazinyl]ethyl]-1,3-dimethylxanthine-amine salt.

### Embodiment 13

A complex compound of any one of Embodiments 1-3, wherein the poly-γ-polyglutamic acid (γ-PGA) derivative includes one selected from a group consisting of an alginate sodium, a poly-γ-polyglutamic acid (γ-PGA), a poly-γ-polyglutamic acid sodium (γ-PGA sodium), and a glutamic acid-L-lysine-L-tyrosine.

### Embodiment 14

A complex compound of any one of Embodiments 1-3, wherein the Co-polymers includes one selected from a group consisting of a hyaluronic acid a polyacrylic acid, a polymethacrylates (PMMA), an Eudragit, a dextran sulfate, a heparan sulfate, a polylactic acid or polylactide (PLA), a polylactic acid sodium (PLA sodium) and polyglycolic acid sodium (PGA sodium).

### Embodiment 15

A pharmaceutical composition of any one of Embodiments 4-6, wherein the halogen atom is one selected from a group consisting of a fluorine, a chlorine, a bromine and an iodine.

### Embodiment 16

A pharmaceutical composition of any one of Embodiments 4-6, wherein the KMUPs amine salt comprising one selected from a group consisting of a KMUP-1-amine salt, a KMUP-2-amine salt, a KMUP-3-amine salt and a KMUP-4-amine salt.

### Embodiment 17

A pharmaceutical composition of any one of Embodiments 4-6, wherein the KMUP-1-amine salt includes a 7-[2-[4-(2-chlorophenyl)piperazinyl]ethyl]-1,3-dimethylxanthine-amine salt.

### Embodiment 18

A pharmaceutical composition of any one of Embodiments 4-6, wherein the KMUP-2-amine salt includes a 7-[2-[4-(2-methoxybenzene)piperazinyl]ethyl]-1,3-dimethylxanthine-amine salt.

### Embodiment 19

A pharmaceutical composition of any one of Embodiments 4-6, wherein the KMUP-3-amine salt includes a 7-[2-[4-(2-nitrobenzene)piperazinyl]ethyl]-1,3-dimethylxanthine-amine salt.

### Embodiment 20

A pharmaceutical composition of any one of Embodiments 4-6, wherein the KMUP-3-amine salt includes a 7-[2-[4-(2-nitrobenzene)piperazinyl]ethyl]-1,3-dimethylxanthine-amine salt.

### Embodiment 21

A pharmaceutical composition of any one of Embodiments 4-6, wherein the poly-γ-polyglutamic acid (γ-PGA) derivative includes one selected from a group consisting of an alginate sodium, a poly-γ-polyglutamic acid (γ-PGA), a poly-γ-polyglutamic acid sodium (γ-PGA sodium), and a glutamic acid-L-lysine-L-tyrosine.

### Embodiment 22

A pharmaceutical composition of any one of Embodiments 4-6, wherein the Co-polymers includes one selected from a group consisting of a hyaluronic acid a polyacrylic acid, a dextran sulfate, and a heparan sulfate.

### Embodiment 23

A method of providing a medical effect for inhibiting MCT-induced pulmonary artery proliferation, the method comprising steps of: providing a subject in need thereof; and administering to the subject in need thereof an effective amount of a pharmaceutical composition comprising the complex compound of any one of Embodiments 1-3.

### Embodiment 24

A method of providing a medical effect for treatment of 5-HT-induced pulmonary artery hypertension, the method comprising steps of: providing a subject in need thereof; and administered to the subject in need thereof an effective amount of the pharmaceutical composition of any one of Embodiments 4-6.

What is claimed is:

1. A complex compound for inhibiting MCT-induced pulmonary artery proliferation, comprising a KMUPs amine salt represented by formula I: 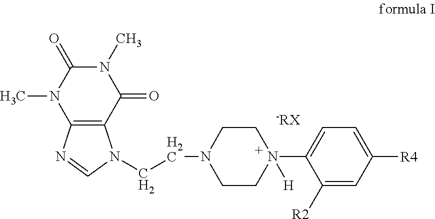
wherein: R2 and R4 are selected independently from a group consisting of a C1˜C5 alkoxy group, a hydrogen, a nitro group, and a halogen atom;
RX includes a carboxylic group selected from a group consisting of a Statin, a sodium carboxyl methylcellulose (sodium CMC), a poly-γ-polyglutamic acid (γ-PGA) derivative and a Co-polymer; and
⁻RX is an anion of a carboxylic group donated from one selected from a group consisting of a Statin, a sodium CMC, a γ-PGA derivative and a Co-polymer.

2. A complex compound as claimed in claim 1, wherein the halogen atom is one selected from a group consisting of a fluorine, a chlorine, a bromine and an iodine.

3. A complex compound as claimed in claim 1, wherein the KMUPs amine salt comprising one selected from a group consisting of a KMUP-1-amine salt, a KMUP-2-amine salt, a KMUP-3-amine salt and a KMUP-4-amine salt.

4. A complex compound as claimed in claim 2, wherein the KMUP-1-amine salt includes a 7-[2-[4-(2-chlorophenyl)piperazinyl]ethyl]-1,3-dimethyl-xanthine-amine salt.

5. A complex compound as claimed in claim 2, wherein the KMUP-2-amine salt includes a 7-[2-[4-(2-methoxybenzene)piperazinyl]ethyl]-1,3-dimethyl-xanthine-amine salt.

6. A complex compound as claimed in claim 2, wherein the KMUP-3-amine salt includes a 7-[2-[4-(4-nitrobenzene)piperazinyl]ethyl]-1,3-dimethyl-xanthine-amine salt.

7. A complex compound as claimed in claim 2, wherein the KMUP-3-amine salt includes a 7-[2-[4-(2-nitrobenzene)piperazinyl]ethyl]-1,3-dimethyl-xanthine-amine salt.

8. A complex compound as claimed in claim 1, wherein the poly-γ-polyglutamic acid (γ-PGA) derivative includes one selected from a group consisting of an alginate sodium, a poly-γ-polyglutamic acid (γ-PGA), a poly-γ-polyglutamic acid sodium (γ-PGA sodium), and a glutamic acid-L-lysine-L-tyrosine.

9. A complex compound as claimed in claim 1, wherein the Co-polymers includes one selected from a group consisting of a hyaluronic acid a polyacrylic acid, a dextran sulfate, a polymethacrylates (PMMA), an Eudragit, a dextran sulfate, a heparan sulfate, a polylactic acid or polylactide (PLA), a polylactic acid sodium (PLA sodium) and a polyglycolic acid sodium (PGA sodium).

10. A pharmaceutical composition for inhibiting MCT-induced pulmonary artery proliferation, comprising an effective amount of a KMUPs amine salt represented by formula I: 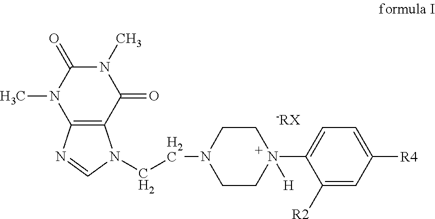
wherein: R2 and R4 are selected independently from a group consisting of a C1˜C5 alkoxy group, a hydrogen, a nitro group, and a halogen atom;
RX includes a carboxylic group selected from a group consisting of a Statin, a sodium carboxyl methylcellulose (sodium CMC), a poly-γ-polyglutamic acid (γ-PGA) derivative and a Co-polymer; and
⁻RX is an anion of a carboxylic group donated from one selected from a group consisting of a Statin, a sodium CMC, a γ-PGA derivative and a Co-polymer.

11. A pharmaceutical composition as claimed in claim 10, wherein the halogen atom is one selected from a group consisting of a fluorine, a chlorine, a bromine and an iodine.

12. A pharmaceutical composition as claimed in claim 10, wherein the KMUPs amine salt comprising one selected from a group consisting of a KMUP-1-amine salt, a KMUP-2-amine salt, a KMUP-3-amine salt and a KMUP-4-amine salt.

13. A pharmaceutical composition as claimed in claim 10, wherein the KMUP-1-amine salt includes a 7-[2-[4-(2-chlorophenyl)piperazinyl]-ethyl]-1,3-dimethyl xanthine-amine salt.

14. A pharmaceutical composition as claimed in claim 10, wherein the KMUP-2-amine salt includes a 7-[2-[4-(2-methoxybenzene)piperazinyl]-ethyl]-1,3-dimethylxanthine-amine salt.

15. A pharmaceutical composition as claimed in claim 10, wherein the KMUP-3-amine salt includes a 7-[2-[4-(4-nitrobenzene)piperazinyl]-ethyl]-1,3-dimethylxanthine-amine salt.

16. A pharmaceutical composition as claimed in claim 10, wherein the KMUP-3-amine salt includes a 7-[2-[4-(2-nitrobenzene)piperazinyl]-ethyl]-1,3-dimethylxanthine-amine salt.

17. A pharmaceutical composition as claimed in claim 10, wherein the poly-γ-polyglutamic acid (γ-PGA) derivative includes one selected from a group consisting of an alginate sodium, a poly-γ-polyglutamic acid (γ-PGA), a poly-γ-polyglutamic acid sodium (γ-PGA sodium), and a glutamic acid-L-lysine-L-tyrosine.

18. A pharmaceutical composition as claimed in claim 10, wherein the Co-polymers includes one selected from a group consisting of a hyaluronic acid a polyacrylic acid, a dextran sulfate a heparan sulfate, a polylactic acid or polylactide (PLA), a polylactic acid sodium (PLA sodium) and a polyglycolic acid sodium (PGA sodium).

19. A method of providing a medical effect for inhibiting MCT-induced pulmonary artery proliferation, the method comprising steps of:
providing a subject in need thereof; and
administering to the subject in need thereof an effective amount of a pharmaceutical composition comprising the complex compound of claim 1.

20. A method of providing a medical effect for treatment of 5-HT-induced pulmonary artery hypertension, the method comprising steps of:
providing a subject in need thereof; and administered to the subject in need thereof an effective amount of the pharmaceutical composition of claim 10.
